(19) United States
(12) Patent Application Publication
(10) Pub. No.: US 2014/0155831 A1
(43) Pub. Date: Jun. 5, 2014

(54) PERIPHERAL VENOUS CATHETER HAVING PROTECTION AGAINST BLOOD SPRAY

(71) Applicants: Ruben Caballero Lopez, Las Palmas de Gran Canaria (ES); Antonio Cornejo Bueno, Cadiz (ES); Manuel Luque Gonzalez, Malaga (ES)

(72) Inventors: Ruben Caballero Lopez, Las Palmas de Gran Canaria (ES); Antonio Cornejo Bueno, Cadiz (ES); Manuel Luque Gonzalez, Malaga (ES)

(21) Appl. No.: 14/232,321

(22) PCT Filed: Jul. 16, 2012
(86) PCT No.: PCT/EP2012/063885

§ 371 Date: Jan. 13, 2014

(30) Foreign Application Priority Data
Jul. 14, 2011 (DE) 20 2011 103 176.8

(51) Int. Cl. A61M 25/06; A61M 39/06

(52) U.S. Cl. CPC A61M 25/0693; A61M 39/06
USPC 604/168.01

(57) ABSTRACT

A peripheral venous catheter comprising a vein indwelling section and a cannula (14), wherein the vein indwelling section includes a connection section and a tube connected to the connection section in a firmly bonded manner, and wherein the cannula is inserted into the vein indwelling section prior to inserting the venous catheter into a vein, and a wall is provided in the connection, which runs substantially perpendicular to the longitudinal extension of the connection section, including a plurality of circular ring-like wall segments, and exhibiting a central passage for the cannula at the middle of the wall, such that the wall segments form a boundary for a chamber provided in the connection section on the side facing the tube, in order to accommodate blood exiting the tube after the cannula has been removed.

### REFERENCE TO RELATED APPLICATION

This application is based upon and claims priority to German Patent Application 20 2011 103 176.8 filed on Jul. 14, 2011.

### BACKGROUND OF INVENTION

1. Field of Invention

The invention relates to a peripheral venous catheter, which includes a vein indwelling section and a cannula. The vein indwelling section includes a connection section and a tube connected to the connection section, in particular, in a firmly bonded manner, and the cannula is inserted into the vein indwelling section before the venous catheter is inserted into a vein.

2. Description of Related Art

Peripheral venous catheters are used daily in the field of health care, especially in hospitals. In particular, they have become an integral when applying entries in the veins of a patient. When applying an entry of this type, the skin of the patient is pierced by the tip of the cannula of the peripheral venous catheter. The cannula is then inserted in the vein together with the tube of the vein indwelling section. After the tube has been inserted, the cannula can be removed. However, certain problems arise for the medical staff upon cannula removal. When extracting the cannula from the vein indwelling section, a spraying or discharge of blood from the vein indwelling section is frequently unavoidable. In particular, due to the danger of infections with dangerous diseases (for example, AIDS), the nursing staff must proceed very cautiously; at this point, the wearing of gloves is frequently required. Nevertheless, after removal of the cannula and prior to the insertion of an infusion tube or a syringe into the vein indwelling section, patients (in particular, other patients) may find the discharge of blood from the vein indwelling section to be unpleasant because they fear an infection by a disease. Further, the discharge of blood from the vein indwelling section leads to an increased material consumption in the hospitals, because the blood must be absorbed with cotton, gauze, or other sterile materials.

A catheter configuration is known, for example, from US 2011/0319825 A1, wherein the configuration includes a catheter flange or connection section, respectively, and a tube. A sealing element is provided in the catheter flange, which is mounted in a displacable manner on an actuator. The sealing element can be displaced axially in relation to the actuator. In a closed position, the catheter flange is fully separated from the tube. In an open position, the sealing element is slid over the actuator in such a manner that the catheter flange is connected to the tube. When inserting a Luer-lock type Luer taper, the sealing element is slid axially from the Luer taper over the actuator. Flaps of a membrane in the sealing element create an aperture, such that fluid can flow through the tube in the catheter flange, or connection section, respectively. However, the catheter configuration known in the art is comparatively complicated in its construction such that there are high costs associated with production. Further, high costs are particularly unacceptable with mass-produced articles such as venous catheters.

### SUMMARY OF THE INVENTION

The present invention addresses the objective of providing a peripheral venous catheter, with which an immediate discharge of blood can be prevented after removal of the cannula from the vein indwelling section, and wherein the venous catheter can be produced in a simple and cost-effective manner. Thus, a wall is provided in the connection section, running substantially perpendicular to the longitudinal extension, which includes a plurality of circular ring-type wall segments, and having a central passage for the cannula in the middle thereof, such that the wall segments form a boundary for a chamber provided in the connection section on the side facing the tube after the cannula has been removed, for receiving blood exiting the tube.

The venous catheter of the present invention, like the catheter known in the art, can also be inserted into the veins, which are pierced by the tip of the cannula, which is inserted in the tube of the vein indwelling section. Thus, the cannula and the tube can be inserted in the vein. After insertion of the catheter, the stiff cannula can then be extracted, in order to ensure greater comfort for the patient while wearing the catheter. Following the extraction of the cannula, the wall segments form a chamber in the vein indwelling section.

The venous catheter of the present invention has the advantage that a chamber can accommodate the blood exiting the tube. Thus, a spraying or discharge of blood immediately after the extraction of the cannula can be prevented, such that a syringe or similar item can be connected to the vein indwelling section, while the blood exiting the tube can be substantially accommodated in the chamber, until the chamber is full. The wall (or, the wall segments) is provided directly in the connection section. Thus, additional sealing elements and actuators known in the prior art are no longer needed. Further, the venous catheter of the present invention can be constructed in a simple and cost-effective manner.

The provision of the central passage in the wall is advantageous, because the cannula can thus be readily inserted in the vein indwelling section by the manufacturer during the initial assembly of the peripheral venous catheter; the cannula need simply be inserted through the central passage of the wall and can then be pushed through the tube. The size of the central passage is preferably selected such that the cannula can be inserted into the vein indwelling section without difficulty, and a discharge of blood through the opening in the middle of the wall can be prevented after removal of the cannula, to the greatest possible extent by the surface tension at the opening. The cannula can be a steel cannula and the tube can be a Teflon tube.

An advantageous design of the peripheral venous catheter provides that the wall segments lie flush and tightly aligned to one another at their abutting edges. This is particularly advantageous because no blood can then exit between the individual wall segments. An exiting of blood is only possible at the central passage in the middle of the wall. Furthermore, it is advantageous if the wall segments are designed such that when inserting a connecting element into the connection section, the wall segments are directly pushed against by the connecting element and give way, in the manner of flaps, toward the inner wall of the connection section. This is advantageous because a connecting element (for example, a syringe) can be inserted into the catheter without any blood escaping, because the catheter or the wall/chamber can accommodate blood exiting the tube until a syringe or similar item has been inserted in the catheter, whereby the wall segments are forced outward by the syringe.

A further advantageous design of the venous catheter is that the connection section exhibits recesses for accommodating the wall segments in its interior, wherein the wall segments give way into the recesses when the connecting element is inserted, such that the connection section exhibits a constant inner diameter over its entire longitudinal extension when the connecting element has been inserted. This has the advantage that the wall segments give way in the manner of flaps when a syringe is inserted, such that they can give way into the recesses, and thus the insertion of a syringe in the opening of the connection section is not impaired by the wall segments. Further, it is advantageous if the wall segments are connected to the connection section by film hinges.

Moreover, it is advantageous if the wall segments are designed such that they do not break when a syringe is inserted into the opening of the connection section. By providing the recesses for accommodating the wall segments and by appropriate material properties of the wall segments, the breaking off of parts of the wall segments during the insertion of a syringe can be prevented, which would otherwise thus allow small fragments to end up in the bloodstream. The material properties can preferably be selected such that a breaking of the wall segments can be prevented, even with a high stress concentration. A breaking of the wall segments can also be prevented during insertion of the syringe by recesses in the interior of the connection section, because a shearing of the wall segments is be prevented as a result of a constant inner diameter obtained by the flap-like giving way of the wall segments.

It is also advantageous if the wall segments are disposed in the distal third of the connection section. This is advantageous because if the wall segments are disposed in the first third of the connection section they can be forced into the recesses by the tip of a syringe. Thus, this enables the introduction of medications and infusions or the removal of blood. At the same time, a certain size of the chamber is obtained through the disposing of the wall segments in the distal third of the connection section, such that this chamber can accommodate a certain blood volume before fluid exits the central opening in the wall.

Another advantageous design of the venous catheter is that the wall segments and the connection section are formed such that the venous catheter can be connected to standard syringes used with the Luer-lock system. By maintaining standard sizes for the standard connections, it is thus possible to insert a majority of the syringes and other connecting elements (known in the art and found in medical supply stores) into the connection section.

Furthermore, it can be provided that the wall segments are made of the same material as the connection section. This leads to production advantages because the wall segments can be molded in the connection section during manufacturing.

Preferably, the venous catheter is designed such that the connection section is made of silicone or polyurethane. This results in advantages with respect to the material properties of the wall segments. The wall segments are not made of a textile or nonwoven, as is the case with membranes that, in the prior art, are disposed on the back surface of the peripheral venous catheter. Instead, the wall segments have the same material properties as the connection section. With the appropriate selection of the material properties, it is therefore possible to prevent fragments of the wall segments (or, entire wall segments) from becoming detached when inserting a syringe in the vein indwelling section, and ending up in the bloodstream of the patient. It is also advantageous if the wall and the connection section are an integrated unit. Moreover, it is advantageous if the wall is glued into the connection section, or is welded to the connection section. In this way, molds used prior to this for the production can be retained. Only a procedural step need be added (specifically, the gluing or welding of the wall to the connection section). Furthermore, it is conceivable that the wall, prior to the insertion of a connecting element, forms an angle of 90° to the inner wall of the connection section.

In one embodiment, the venous catheter is designed such that the wall exhibits a hardness in the range of 0-100 Shore, preferably in the range of 30-70 Shore, and more preferably in the range of 40-60 Shore. The hardness is selected such that the wall does not break when a syringe is inserted. Rather, the wall segments remain attached to the inner wall of the connection section by film hinges, and can give way into the recesses provided for this.

Furthermore, it may be provided that the wall exhibits a thickness in the range of 0.0001-3 mm, preferably in the range of 0.001-2 mm, and more preferably in the range of 0.01-1 mm. The thickness of the wall is also selected such that the insertion of a medical instrument (for example, a syringe) does not require a great deal of strength, and the wall is not destroyed when extracting the cannula after the venous catheter has been inserted.

Further advantages and designs of the invention can be derived from the following description, based on which an embodiment example of the invention is described and explained in greater detail.

### BRIEF DESCRIPTION OF THE DRAWINGS

FIG. 1: a side view of a peripheral venous catheter,

FIG. 2: a section of a vein indwelling section of a venous catheter according to the invention,

FIG. 3: a cutaway of a side view of the vein indwelling section of FIG. 2, and

FIG. 4: a vein indwelling section with a syringe inserted therein.

### DETAILED DESCRIPTION OF THE INVENTION

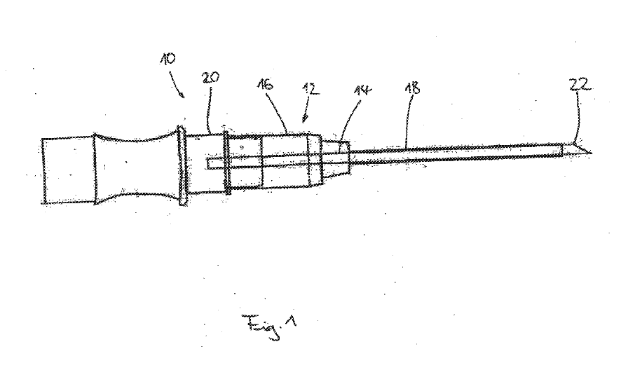

FIG. 1 depicts a venous catheter as known from the art. A peripheral catheter 10 is depicted, which includes a vein indwelling section 12 and a cannula 14. The vein indwelling section 12 includes a connection section 16 and a tube 18. The cannula 14 is mounted in a mounting support 20. Prior to inserting the peripheral venous catheter 10, the cannula 14 is inserted in the vein indwelling section 12 such that the cannula 14 pierces the tube 18. The tip 22 of the cannula extends beyond the tube 18. As a result, when inserting the catheter 10, the vein of the patient can be pierced by the tip 22 of the cannula 14, and the tube 18 of the vein indwelling section 12 can be inserted together with the cannula 14 into the vein. The cannula 14 can be a steel cannula thereby, wherein the tube 18 can be a Teflon tube.

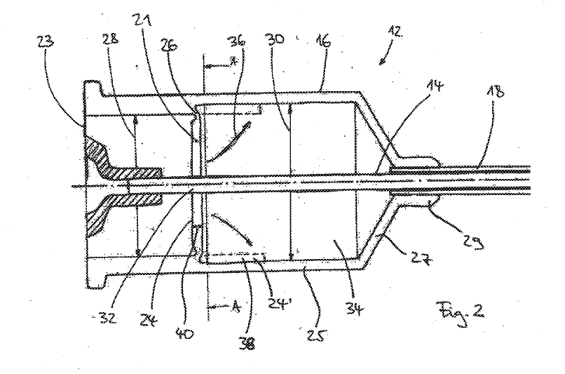

FIG. 2 shows a vein indwelling section 12 of a venous catheter 10 according to the invention. The components corresponding to the components in FIG. 1 are indicated with the corresponding reference symbols. The vein indwelling section 12 includes a connection section 16 and a tube 18. The cannula 14 is inserted into the vein indwelling section 12. The cannula 14 pierces the connection section 16 and the tube 18 thereby. The cannula 14 is likewise mounted in a mounting support 20. The connection section 16 is connected to the tube 18 in a firmly bonded manner such that no blood can exit between the tube 18 and the connection section 16. The connection section 16 has an opening 23 at its distal end. A wall 21 having wall segments 24 is disposed in the interior of the connection section 16. The wall segments 24 are designed as circular ring sections.

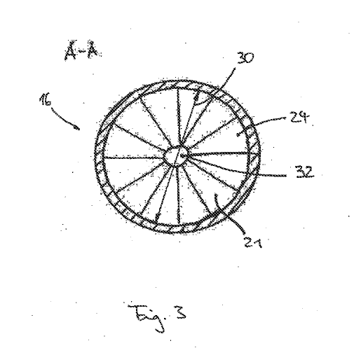

FIG. 3 shows a cutaway section of the side view of a connection section 16 of FIG. 2. The circular ring section shaped wall segments 24 are clearly shown. In the connection section 16 depicted in FIG. 2, the wall segments 24 are attached in the connection section 16 by film hinges 26. The connection section 16 has a substantially cylindrical main section 25 and a conical end section 27, wherein the conical end section transitions into a likewise cylindrical tube connection section 29, at which the connection section 16 is connected to the tube 18. The connection section 16 exhibits two different diameters 28, 30 in its interior. The diameter 28 is smaller than the diameter 30 thereby. The cannula 14 pierces the wall segments 24 in the middle, in a central passage 32. This central passage 32 is likewise visible in a cutaway view in FIG. 3.

FIG. 2 shows the vein indwelling section 12 with an inserted cannula 14 prior to the insertion of the venous catheter 10 in the vein of the patient. After the venous catheter 10 has been inserted, the cannula 14 is removed from the vein indwelling section 12. Thus, only the tube 18 remains in the vein. The wall segments 24 form a chamber 34 in the distal third of the connection section 16. This chamber 34 can accommodate blood exiting from the tube 18, and thus prevent an immediate exit of blood from the vein indwelling section 12. If a connecting element (for example, a syringe) is inserted in the vein indwelling section 12, the wall segments 24 give way in the direction of the arrow 36, into the recesses 38. The recesses are obtained in that the diameter 30 is larger than the diameter 28. The wall segments 24′ are indicated in FIG. 2 in their displaced position by a broken line. The diameter 30 is larger than the diameter 28 by twice the thickness 40 of the wall segments 24. Thus, the connection section 16 still exhibits a constant diameter 28 over its longitudinal extension when a connecting element has been inserted, because the wall segments 24′ are then located in the recesses 38. Because of the constant diameter 28, the insertion of connecting elements (for example, a syringe) can be facilitated. The diameter 28 is selected such that standard connecting elements used in the Luer-lock system can be inserted without difficulty in the connection section 16. Because the wall segments 24 are disposed in the distal third of the connection section 16, they can be readily forced out of the connecting elements and give way into their displacement position 24′.

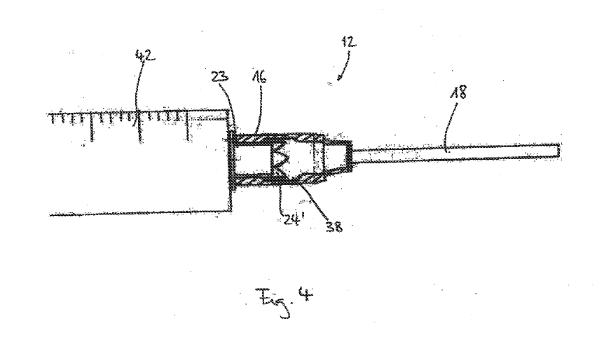

FIG. 4 depicts a situation in which a connecting element has been inserted in the vein indwelling section 12. A syringe 42 is inserted in the opening 23 of the connection section 16. The wall segments 24′ are located in the recesses 38.

1. A peripheral venous catheter comprising a vein indwelling section and a cannula, wherein the vein indwelling section includes a connection section and a tube connected to the connection section in a firmly bonded manner, and wherein the cannula is inserted into the vein indwelling section prior to inserting the venous catheter into a vein, the connection section is made of silicone or polyurethane, and a wall is provided in the connection section, which runs substantially perpendicular to the longitudinal extension of the connection section, including a plurality of circular ring-like wall segments, and exhibiting a central passage for the cannula at the middle of the wall, such that the wall segments form a boundary for a chamber provided in the connection section on the side facing the tube, in order to accommodate blood exiting the tube after the cannula has been removed, and the wall and the connection section are an integrated unit.

2. The peripheral venous catheter as set forth in claim 1, wherein the wall segments lie flush and tightly aligned to one another at their abutting edges.

3. The peripheral venous catheter as set forth in claim 1, wherein a connecting element is inserted into the connection section, such that the wall segments are directly pushed against by the connecting element and give way toward the inner wall of the connection section in the manner of flaps.

4. The peripheral venous catheter as set forth in claim 1, wherein the connection section exhibits recesses in its interior for accommodating the wall segments, wherein the wall segments give way into the recesses when the connecting element is inserted, such that the connection section exhibits a constant inner diameter over its entire longitudinal extension when a connecting element has been inserted.

5. The peripheral venous catheter as set forth in claim 1, wherein the wall segments are connected to the connection section via film hinges.

6. The peripheral venous catheter as set forth in claim 1, wherein the wall segments are designed such that they do not break when inserting a syringe into the opening of the connection section.

7. The peripheral venous catheter as set forth in claim 1, wherein the wall segments are disposed in the distal third of the connection section.

8. The peripheral venous catheter as set forth in claim 1, wherein the wall segments and the connection section are designed such that the venous catheter can be connected to standard syringes according to the Luer-lock system.

9. The peripheral venous catheter as set forth in claim 1, wherein the wall segments are made of the same material as the connection section.

10. (canceled)

11. (canceled)

12. The peripheral venous catheter as set forth in claim 1, wherein the wall is glued into the connection section.

13. The peripheral venous catheter as set forth in claim 1, wherein the wall forms an angle of 90° to the inner wall of the connection section, prior to the insertion of a connecting element.

14. The peripheral venous catheter as set forth in claim 1, wherein the wall exhibits a hardness in the range of 0-100 Shore.

15. The peripheral venous catheter as set forth in claim 1, wherein the wall exhibits a thickness in the range of 0.0001-3 mm.

16. The peripheral venous catheter as set forth in claim 1, wherein the wall is welded to the connection section.

17. The peripheral venous catheter as set forth in claim 1, wherein the wall exhibits a hardness in the range of 30-70 Shore.

18. The peripheral venous catheter as set forth in claim 1, wherein the wall exhibits a hardness in the range of 40-60 Shore.

19. The peripheral venous catheter as set forth in claim 1, wherein the wall exhibits a thickness in the range of 0.001-2 mm.

20. The peripheral venous catheter as set forth in claim 1, wherein the wall exhibits a thickness in the range of 0.01-1 mm.
